(19) United States
(12) Patent Application Publication
(10) Pub. No.: US 2010/0174243 A1
(43) Pub. Date: Jul. 8, 2010

(54) Apparatus for Delivery of Therapeutic Material to an Intervertebral Disc and Method of Use

(75) Inventor: William F. McKay, Memphis, TN (US)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedic, Inc., Warsaw, IN (US)

(21) Appl. No.: 12/348,568

(22) Filed: Jan. 5, 2009

(51) Int. Cl. A61M 25/00; A61M 5/32; A61M 31/00; A61F 2/44

(52) U.S. Cl. 604/264; 604/272; 604/278; 604/500; 623/17.16

(57) ABSTRACT

An apparatus, for introducing fluent therapeutic material through an annulus fibrosus of an intervertebral disc, includes a needle having a distal end for insertion through an entry site of the annulus fibrosus. The needle defines a first conduit through which the pressurized fluent therapeutic material is introduced. An expandable seal is disposed about an exterior surface of the needle. The expandable seal has a non-expanded, non-sealing configuration to facilitate penetration of the needle through the annulus fibrosus and withdrawal of the needle therefrom. The expandable seal also has an expanded, sealing configuration during introduction of the fluent therapeutic material. The expandable seal then engages the annulus fibrosus in its expanded, sealing configuration to prevent or minimize leakage of fluent therapeutic material through the entry site. The expandable seal assumes its expanded, sealing configuration during introduction of fluent therapeutic material under pressure into the annulus fibrosus.

### TECHNICAL FIELD

The present disclosure generally relates to medical devices and methods for the treatment of spinal disorders, and more particularly to methods and apparatus for treating intervertebral discs.

### BACKGROUND

Any publications or references discussed herein are presented to describe the background of the invention and to provide additional detail regarding its practice. Nothing herein is to be construed as an admission that the inventors are not entitled to antedate such disclosure by virtue of prior invention.

Intervertebral discs lie between adjacent vertebrae of a spine. An intervertebral disc includes fibrosus bands, which provide cushion to facilitate motion of the vertebrae and spacing of the vertebrae from nerves and vessels. The fibrosus bands include an outer annulus fibrosus, which is the peripheral portion of an intervertebral disc and defines an inner nucleus pulposus. The nucleus pulposus includes loose fibers suspended in a gel substance having a jelly like consistency that absorbs impacts to the body while keeping the vertebrae separated.

The annulus fibrosus is susceptible to tearing due to injury and/or the aging process. Tears in the annulus fibrosus may cause disc herniation whereby the nucleus pulposus, which can include inflammatory cytokines, leaks or migrates from the intervertebral disc into the body. Typically, the herniated disc compresses the nerves and/or other vessels resulting in chronic and debilitating pain. Further, the leakage of nucleus pulposus may contact the nerves, which can cause significant pain.

Non-surgical treatments, such as medication, rehabilitation and exercise can be effective, however, may fail to relieve the symptoms associated with the above conditions. Surgical treatment of spinal disorders can include discectomy, laminectomy, fusion and implantable prosthetics. Other known surgical treatments include implanting a balloon in the disc space and filling the balloon with a polymer. Other treatments require forcing apart vertebrae with a balloon so that a thermoplastic material can be injected into the disc space. These treatments, however, may suffer from disadvantages and drawbacks. For example, the devices employed with these treatments do not prevent extrusion of materials through the intervertebral disc during injection. Further, these treatments may not effectively fill the tears in the intervertebral disc.

Therefore, it would be desirable to provide an apparatus and method for treating intervertebral discs that includes a needle for introducing fluent therapeutic material into the intervertebral disc, which includes an expandable seal that pressurizes the intervertebral disc and forms a seal with the surrounding disc tissue to prevent or minimize leakage of the fluent therapeutic material during treatment. Desirably, the apparatus prevents or minimizes leakage through the annulus fibrosus with an expandable seal having an expandable, sealing configuration during introduction of the fluent therapeutic material under pressure into the intervertebral disc space. It would be most desirable if the expandable seal is fabricated from a soft polymer such that the sealing configuration includes friction and/or sealing characteristics with the surrounding disc tissues.

### SUMMARY OF THE INVENTION

Accordingly, the present disclosure provides an apparatus and method for treating an intervertebral disc that includes a needle for introducing fluent therapeutic material, which includes an expandable seal that pressurizes the disc and forms a seal with the surrounding disc tissue to prevent or minimize leakage of the fluent therapeutic material and/or other body fluids during treatment. Desirably, the apparatus prevents or minimizes leakage through the annulus fibrosus with an expandable seal having an inflatable sealing configuration during introduction of the fluent therapeutic material under pressure into the intervertebral disc space. Most desirably, the expandable seal is fabricated from a soft polymer such that the sealing configuration includes friction and/or sealing characteristics with the surrounding disc tissues.

In one embodiment, the present disclosure includes a needle to pressurize the intervertebral disc while injecting it with therapeutic agents. The needle design has a circumferential inflatable balloon on a mid-portion of the needle that is inflated after it is inserted into the disc space. The balloon serves several purposes, including, for example preventing an injected polymer from extruding through the disc during injection, pressurizing the intervertebral disc to force an injectable polymer deep into the tears and crevasses within the disc, facilitating disc filling to provide improved disc mechanical stabilization, facilitating improved mechanical interlocking of the solidified polymer within the disc, and facilitating sealing of the annulus tears that leak inflammatory cytokines. Upon filling of the disc with the injectable polymer, the balloon is deflated and the needle withdrawn. It is contemplated that the balloon is not required to be filled with polymer.

Desirably, the balloon is flush with the outer diameter of the needle to facilitate insertion into the intervertebral disc. The needle can be inserted a sufficient depth into the disc so that when the balloon is inflated, the surrounding disc tissue holds the balloon in place and forms a seal. Preferably, the needle is inserted such that the balloon is positioned just inside the nucleus pulposus space such that as the balloon is inflated, the balloon pressurizes the nucleus pulposus where the injectable polymer will be injected. It is envisioned that the balloon is inflated via a separate channel disposed with the needle, which can be located with the interior of the needle or exterior to the needle.

The balloon can be fabricated from materials having strength and flexibility characteristics according to the requirements of a particular application, such as polyvinylchloride, polyethylene terephthalate (PET), nylon, polyurethane, silicone, thermoplastic rubber, nylon, and thermoplastic elastomer materials. See, for example, U.S. Pat. Nos. 4,969,888, 5,827,289, 5,972,015, 6,235,043, 6,248,110, and 6,607,544. It is contemplated that the balloon is not required to be fabricated from an implantable polymer. Desirably, the balloon is fabricated from a soft polymer with friction and/or sealing capabilities with the surrounding tissues of a body. It is envisioned that the balloon is fabricated from a material that does not adhere to a polymer being injected for the treatment of the intervertebral disc so that the balloon can be deflated and removed from the disc space following injection of the treatment polymer.

The design of the present disclosure facilitates flow of an injectable polymer into the void spaces of an intervertebral disc. This configuration provides for disc stabilization and prevents leaking of inflammatory cytokines from a degenerated intervertebral disc. It is contemplated that such design can be employed with a treatment for overcoming back and leg pain. As such, the apparatus and method including the needle of the present disclosure is employed to provide a more effective therapy for back and leg pain.

In one particular embodiment, in accordance with the principles of the present disclosure, an apparatus for introducing fluent therapeutic material through an annulus fibrosus of an intervertebral disc is provided. The apparatus includes a needle having a distal end for insertion through an entry site of the annulus fibrosus. A proximal end is coupled to a source of pressurizable fluent therapeutic material. The needle defines a first conduit through which the pressurized fluent therapeutic material is introduced. An expandable seal is disposed about an exterior surface of the needle. The expandable seal has a non-expanded, non-sealing configuration to facilitate penetration of the needle through the annulus fibrosus and withdrawal of the needle therefrom. The expandable seal also has an expanded, sealing configuration that may be used during introduction of the fluent therapeutic material. The expandable seal then engages the annulus fibrosus in its expanded, sealing configuration to prevent or minimize leakage of fluent therapeutic material back through the entry site. The expandable seal assumes its expanded, sealing configuration during introduction of fluent therapeutic material and may be collapsed following the introduction of the fluent therapeutic material. Optionally, fluent therapeutic material is a settable material, where the expandable seal may be collapsed when the settable material is set to a desirable degree.

The expandable seal may be expanded using a fluid in connection with the expandable seal. For example, the needle may have a second conduit communicating with the expandable seal wherein the second conduit is configured to introduce a pressurized inflating medium comprising a gas or fluid from a distal source through the second conduit and into the expandable seal. The second conduit is also configured to convey the pressurized inflating medium from the expanded seal, thereby allowing the expandable seal to assume a non-expanded, non-sealing configuration.

The expandable seal in its non-expanded, non-sealing configuration can be disposed in a circumferential recess in the exterior surface of the needle. For example, an exposed surface of the expandable seal may be substantially flush with the exterior surface of the needle. The expandable seal, in the expanded, sealing configuration, may have a substantially toroidal configuration.

The expandable seal, in its expanded, sealing configuration is dimensioned such that it occupies not more than about 20% of a volume contained by the annulus fibrosus, and the expandable seal does not occupy more than about 5% of a volume contained by the annulus fibrosus.

The needle may further include at least one guide mark on its exterior surface to indicate a distance of insertion of the needle through the annulus fibrosus, wherein the expandable seal will be within the nucleus pulposus but in close proximity to the annulus fibrosus. The needle may also include a stop affixed to the exterior surface. The stop is configured to prevent over-insertion of the needle through the annulus fibrosus.

In another embodiment, a method for introducing fluent therapeutic material into the annulus fibrosus of an intervertebral disc is provided. The method includes the steps of: inserting a needle, similar to that described herein, through an entry site of the annulus fibrosus and into a nucleus pulposus of the intervertebral disc; inflating the expandable seal with pressurized inflating medium whereby the expandable seal assumes its inflated, sealing configuration such that the expandable seal engages the annulus fibrosus to prevent or minimize flow of the fluent therapeutic material through the entry site during the introduction of the fluent therapeutic material into the nucleus pulposus and/or annulus fibrosus; introducing the fluent therapeutic material under pressure into the nucleus pulposus and/or annulus fibrosus while the seal is in its inflated, sealing configuration; deflating the expandable seal to its non-expanded, non-sealing configuration; and removing the needle. Optionally, the method includes introducing a flowable, settable therapeutic material and/or retaining the expandable seal in the expanded configuration until the settable therapeutic material has assumed a viscosity or set sufficient to prevent or reduce extrusion of the therapeutic material through the channel left after removal of the needle.

The method may further include the steps of: releasing pressurized inflating medium from the expandable seal following the introduction of the fluent therapeutic material into the space defined by displacement of the nucleus pulposus and/or annulus fibrosus whereby the expandable seal assumes its uninflated, non-sealing configuration; and withdrawing the needle from the space defined by displacement of the nucleus pulposus and annulus fibrosus while the expandable seal is in its uninflated, non-sealing configuration.

### BRIEF DESCRIPTION OF THE DRAWINGS

The features of the present disclosure will become more readily apparent from the specific description accompanied by the following drawings, in which:

FIG. 1 is a side view of one particular embodiment of an apparatus in accordance with the principles of the present disclosure;

FIG. 2 is a cutaway, side cross section view of a distal end of the apparatus shown in FIG. 1, with an expandable member in a non-expanded, non-sealing configuration;

FIG. 3 is section view of a needle of the apparatus taken along lines 3-3 of FIG. 1;

FIG. 4 is a cutaway, side view of the distal end of the apparatus shown in FIG. 1, with an expandable member in an expanded, sealing configuration;

FIG. 5 is a cutaway, side cross section view of the distal end of the apparatus shown in FIG. 1, with an expandable member in an expanded, sealing configuration; and

FIG. 6 is plan view of the apparatus shown in FIG. 1 being employed for treatment of an intervertebral disc in accordance with the principles of the present disclosure.

Like reference numerals indicate similar parts throughout the figures.

### DETAILED DESCRIPTION OF THE INVENTION

The exemplary embodiments of the apparatus and methods of the subject invention are discussed in terms of medical devices and methods for the treatment of spinal disorders and more particularly, in terms of an apparatus and method for treating intervertebral discs that includes a needle for introducing fluent therapeutic material into the intervertebral disc. The needle has an expandable seal that pressurizes the intervertebral disc and forms a seal with the surrounding disc tissue to prevent or minimize leakage of a fluent therapeutic material during treatment. It is envisioned that the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. It is further envisioned that the present disclosure may be employed with surgical treatments including open surgery and minimally invasive procedures, of such disorders, such as, for example, discectomy, laminectomy, fusion, bone graft and implantable prosthetics.

It is contemplated that the present disclosure may be employed with other osteal and bone related applications, including those associated with diagnostics and therapeutics. It is further contemplated that the apparatus and methods of the subject invention may be employed in a surgical treatment with a patient in a prone or supine position, employing a posterior, lateral or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention.

Also, as used in the specification and including the appended claims, the singular forms “a,” “an,” and “the” include the plural, and reference to a particular numerical value includes at least that particular value, unless the context clearly dictates otherwise.

Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

All methods described herein may be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

As used herein, “comprising,” “including,” “containing,” “characterized by,” and grammatical equivalents thereof are inclusive or open-ended terms that do not exclude additional, unrecited elements or method steps, but will also be understood to include the more restrictive terms “consisting of” and “consisting essentially of.”

All publications, patents and patent applications cited in this specification are herein incorporated by reference in their entirety as if each individual publication, patent or patent application were specifically and individually indicated to be incorporated by reference.

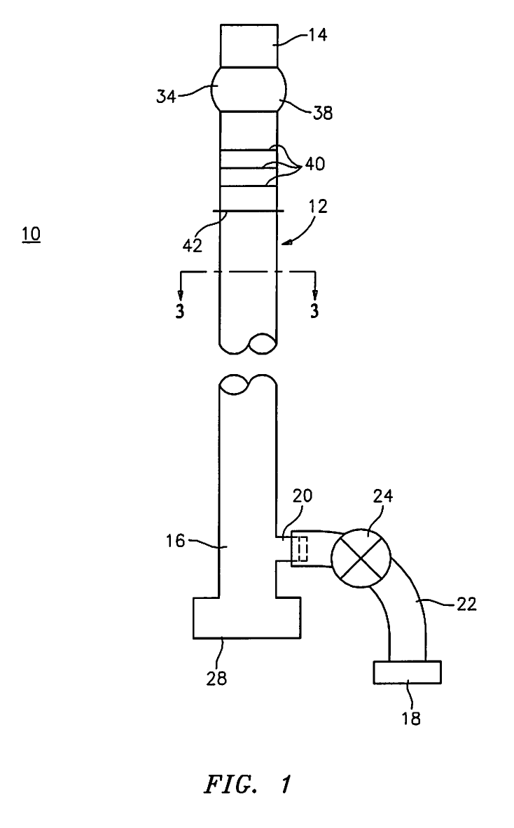

The following discussion includes a description of the apparatus of the subject invention, and related components and exemplary methods of employing the apparatus of the subject invention. Alternate embodiments are also disclosed. Reference will now be made in detail to an exemplary embodiment of the present disclosure, which is illustrated in the accompanying figures. Turning now to FIGS. 1-3, the components of an apparatus 10 and related methods of use, in accordance with the principles of the present disclosure, are illustrated.

The components of apparatus 10 can be fabricated from materials suitable for medical applications, including metals, polymers, ceramics, biocompatible materials and/or their composites, and combinations thereof, depending on the particular application and/or preference of a medical practitioner. The components of apparatus 10 may include radiolucent and/or radio opaque materials.

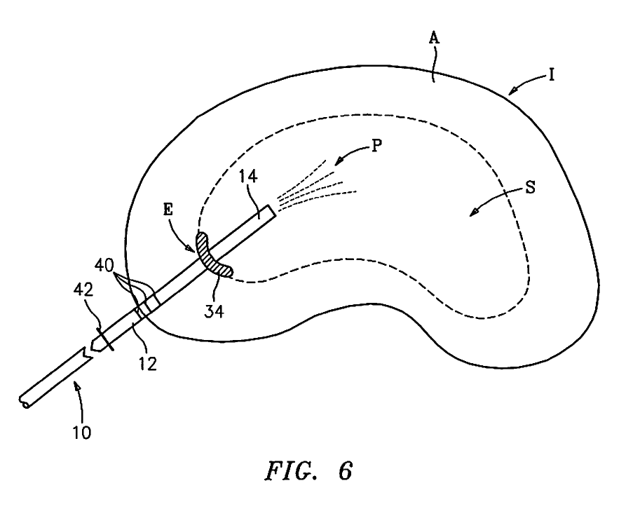

Apparatus 10 includes a needle 12 configured for introducing fluent therapeutic material, such as, for example, an injectable polymer P, through an annulus fibrosus A of an intervertebral disc I (FIG. 6). Needle 12 enters a space S formed by the displacement of nucleus pulposus from, for example, cracks and tears in annulus fibrosus A, or from degeneration of the nucleus pulposus, to facilitate treatment and repair of intervertebral disc I, as discussed in more detail below.

It is envisioned that the fluent therapeutic material is sufficiently flowable when introduced through annulus fibrosus A and into space S. The fluent therapeutic material may remain fluent after introduction under pressure, or may polymerize, cure, or otherwise harden to a less fluent or nonflowable state. In a preferred embodiment of the subject invention, the fluent therapeutic material is PVA hydrogel.

It is contemplated that the fluent therapeutic material is a biologically acceptable material and may include materials effective to promote healing, repair, regeneration or restoration of an intervertebral disc or to facilitate proper disc function. For example, the fluent therapeutic material may include culture media, cells, pharmaceuticals such as, for example, steroids, antibiotics, anti-inflammatory medications, immunosuppressive medications, tissue necrosis factor alpha or its antagonists and analgesics, tissue growth or differentiation factors, such as, for example, recombinant generated morphogenic proteins including but not limited to BMP, including BMP-2 and BMP-7, GDF, including GDF-5, PDGF, TGF-β, EGF/TGF-α, IGF-1, βPFGF, genes or gene vectors in solution, biologic materials such as, for example, hyaluronic acid, non-cross linked collagen, fibrin, silk, elastin, polyglycolic acid, polylactic acid, polytetrafluoroethylene, liquid fat or oils, synthetic polymers, such as, for example, polyethylene, polyethylene glycol, liquid silicones, synthetic oils, curable hydrogels and saline. One skilled in the art will understand that any of these materials may be used alone or that a combination of two or more of these materials may be used together to form the fluent therapeutic material. Moreover, additional additives such as thickening agents, carriers, polymerization initiators or inhibitors may also be included, depending on the desired infusion and long-term performance characteristics.

Needle 12 delivers injectable polymer P to space S while preventing or minimizing leakage or flow of the injectable polymer from space S through an entry site during delivery. It is envisioned that apparatus 10 may be employed with other medical applications, as referred to herein.

Needle 12 has a distal end 14 for insertion through an entry site E (FIG. 6), for example, an annulus fibrosus A. A proximal end 16 is coupled to a source 28 of a pressurizable injectable polymer P (FIG. 6). It is contemplated that reference to the needle of apparatus 10 in the description and claims of the present disclosure includes structure for introducing fluent therapeutic material to intervertebral disc I such as cannulas, trocars, sheaths, minimally invasive instruments and other structure that defines a conduit configured for the passage of fluids. It is envisioned that needle 12 can vary in length and cross section according to the requirements of a particular application. Distal end 14 may include a nozzle, tapered cutting edge or blunt tip. Distal end 14 and proximal end 16 may have uniform or alternate cross section size and geometry. Needle 12 may be fabricated from biologically acceptable materials such as metals, polymerics and ceramics. It is envisioned that needle 12 is fabricated from stainless steel, cobalt chromium, titanium, nitinol and alloys or mixtures thereof. It is further envisioned that needle 12 or portions thereof may be flexible, semi-rigid, rigid and/or variations thereof.

Needle 12 defines a first conduit, such as, for example, first lumen 26 through which injectable polymer P is introduced through entry site E to space S. First lumen 26 is connected to source 28, which is in fluid connection to the proximal end 16. First lumen 26 is configured for flow of injectable polymer P. The cross section of first lumen 26 can vary in size and geometry according to the requirements of a particular application. Source 28 may be a syringe barrel with plunger, pressurized container and/or wall connection. The flow of injectable polymer P may be regulated and/or valve controlled manually, electronically or processor controlled, as is known to one skilled in the art. It is envisioned that needle 12 may include one or a plurality of conduits through which injectable polymer P is introduced through entry site E to space S. It is further envisioned that the pressurized injectable polymer P is introduced at a pressure in the range of 1-120 psi. The pressurized flow may be constant or varying, depending on the application.

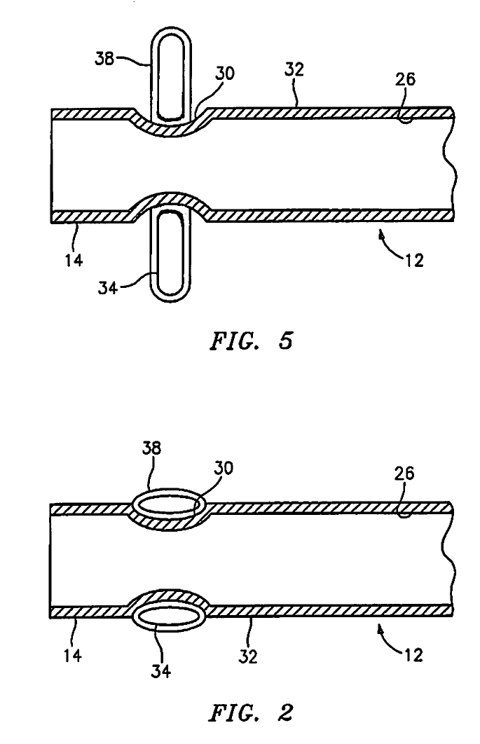

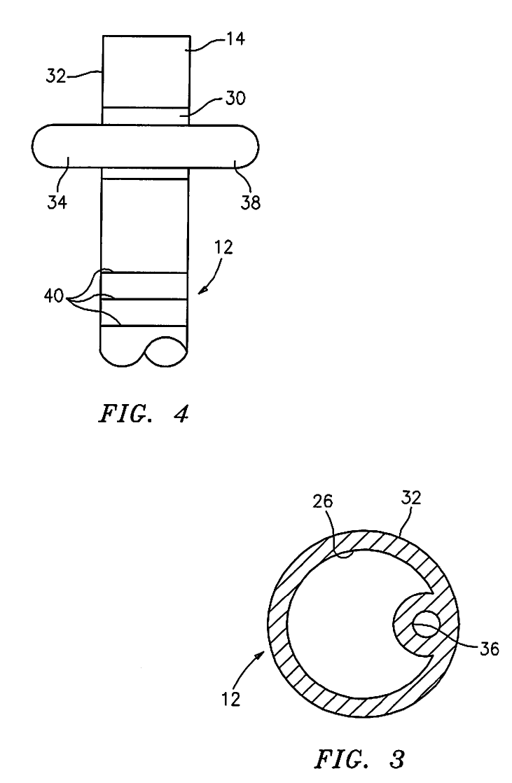

Needle 12 defines a circumferential recess 30 in an exterior surface 32 adjacent distal end 14. An expandable seal, such as, for example, an inflatable sealing member 34 is configured for disposal about exterior surface 32 and in circumferential recess 30. Inflatable sealing member 34 is expandable and can be contracted, between a non-expanded or non-inflated, non-sealing configuration, as shown in FIG. 2, and an expanded or inflated, sealing configuration, as shown in FIGS. 4 and 5. It is contemplated that the expandable seal may include alternate or combinations of an expanding structure such as balloons, expanding arms, flexible wire, expanding linkages, tongs, expanding bands and articulating linkages.

Needle 12 defines a second conduit, such as, for example, second lumen 36 communicating with inflatable sealing member 34. Second lumen 36 is connected to a source 18 of pressurized expanding medium, such as, for example, inflating air, gas, or a fluid, adjacent proximal end 16. Second lumen 36 is configured to introduce the pressurized air from source 18 into inflatable sealing member 34 and to convey the pressurized air from inflatable sealing member 34 whereby inflatable sealing member 34 assumes its non-inflated, non-sealing configuration. The pressurized air is introduced at a pressure in a range of 1-20 psi or at a pressure sufficient to retain inflation during injection of the polymer P. The pressurized air flow may be constant or varied, depending on the application. It is contemplated that alternative pressurized expanding mediums may be employed such as sterile water or saline. It is further contemplated that needle 12 defines a third conduit communicating with inflatable sealing member 34 and is configured to convey pressurized medium from inflatable sealing member 34 whereby inflatable sealing member 34 assumes its non-inflated, non-sealing configuration.

The cross section of second lumen 36 can vary in size and geometry according to the requirements of a particular application. Source 18 may be a syringe barrel with plunger, pressurized container and/or wall connection. The flow and/or pressure may be regulated and/or valve controlled manually, electronically or processor controlled, as is known to one skilled in the art. It is envisioned that needle 12 may include one or a plurality of conduits through which pressure may be introduced and conveyed. Proximal end 16 includes a fitting 20 attached with a supply conduit 22 connected to source 18.

In the non-inflated, non-sealing configuration, inflatable sealing member 34 facilitates penetration of needle 12 through annulus fibrosus A and withdrawal of needle 12 from annulus fibrosus A. An exposed surface 38 of inflatable sealing member 34 is in a substantially flush alignment with exterior surface 32 in the non-inflated, non-sealing configuration.

In the inflated, sealing configuration of inflatable sealing member 34, needle 12 introduces injectable polymer P to space S. Inflatable sealing member 34 has a substantially toroidal configuration and engages annulus fibrosus A adjacent entry site E to prevent or minimize leakage of injectable polymer P through entry site E. The disc tissue surrounding entry site E maintains inflatable sealing member 34 in position during introduction of injectable polymer P to space S. Inflatable sealing member 34 is disposed just inside of space S to form a seal with the disc tissue. During introduction of injectable polymer P under pressure into annulus fibrosus A, inflatable sealing member 34 assumes its inflated, sealing configuration. It is contemplated that inflatable sealing member 34, in its inflated, sealing configuration, may have alternative configurations such as spherical, cube, dog bone or polygonal.

Inflatable sealing member 34 is configured and dimensioned such that, in the inflated sealing configuration, it does not occupy more than about 20% of space S and/or a volume contained by annulus fibrosus A. It is envisioned that inflatable sealing member 34, in its inflated, sealing configuration is configured and dimensioned such that it does not occupy more than about 5% of space S and/or a volume contained by annulus fibrosus A. In another exemplary embodiment, the inflatable sealing member 34 assumes a diameter, when in its fully extended, sealing configuration, that is not more than about 30%, 25%, 20%, 15%, 10%, 5%, or 2% relative to the diameter of the needle 12. It is contemplated that injectable polymer P may be introduced at a greater, equal or lower pressure than the pressure at which the pressurized air is introduced.

It is envisioned that inflatable sealing member 34 is fabricated from biologically acceptable materials including vinyl, polyvinyl chloride, silicone, nylon, thermoplastic rubbers, thermoplastic elastomer materials, polyethylenes, ionomer, polyurethane, polyolefins, polyetheretherketone, polyactide, polyglyclolide, poly(lactide-co-glycolide), poly(dioxanone), poly(ε-caprolactone), poly(hydroxylbutyrate), poly(hydroxylvalerate), tyrosine-based polycarbonate, polypropylene fumarate, polyethylene tetraphthalates (PET), or combinations thereof. Inflatable sealing member 34 may be constructed of materials to achieve various desired characteristics such as biocompatibility, strength, thickness, rigidity, elasticity, durability, permeability.

Distal end 14 of needle 12 includes guide marks 40 on exterior surface 32 to indicate a distance of insertion of needle 12 through annulus fibrosus A. Guide marks 40 may include radio opaque or radiolucent material for identification of needle 12 depth with x-ray or similar. Needle 12 may include one or a plurality of guide marks. Needle 12 includes a stop 42 disposed with exterior surface 32, which is configured to prevent over insertion of needle 12 through annulus fibrosus A. It is contemplated that needle 12 may not include guide marks 40 and/or stop 42.

Referring to FIG. 6, apparatus 10, similar to that discussed above, is employed with a method for introducing a fluent therapeutic material, such as injectable polymer P into annulus fibrosus A of intervertebral disc I. Intervertebral disc I suffers from damage, for example, cracking and/or tearing, such that nucleus pulposus is leaking or migrating therefrom. Needle 12 is inserted through entry site E and into space S. It is envisioned that such treatment can include treatment to promote healing, repair, regeneration or restoration of intervertebral disc I or to facilitate proper disc function, and other treatments discussed herein.

Needle 12 is introduced into the soft tissue of a patient to the targeted intervertebral disc I. Distal end 14 of needle 12, with inflatable sealing member 34 in its uninflated, non-sealing configuration, penetrates annulus fibrosus to a desired depth within space S. Guide marks 40 facilitate location of distal end 14 within intervertebral disc I, as described above. Stop 42 prevents over penetration of needle 12. Needle 12 is inserted into space S such that inflatable sealing member 34 is disposed adjacent to and in engagement with an interior wall of annulus fibrosus A.

Inflatable sealing member 34 is inflated with pressurized expanding medium such as air, biologically compatible gas or fluid as discussed above, such that inflatable sealing member 34 assumes its inflated sealing configuration. Inflatable sealing member 34 engages the surrounding disc tissue, including the interior wall of annulus fibrosus A adjacent entry site E to create a seal between the components of apparatus 10, for example, needle 12 and inflatable sealing member 34, and the disc tissue surrounding entry site E. This configuration prevents or minimizes flow of injectable polymer P back through entry site E during introduction of injectable polymer P into space S.

In one embodiment, the toroidal configuration of inflatable sealing member 34 is positioned proximate to the interior wall of annulus fibrosus A, thereby facilitating formation of an annular seal with the disc tissue surrounding entry site E, which prevents or minimizes leaking of injectable polymer P and/or other fluids. The seal formed by inflatable sealing member 34 also prevents or minimizes leakage or migration of inflammatory cytokines and other material from space S. It is envisioned that inflatable sealing member 34 is fabricated from a material having adhesive properties to facilitate the seal formation with the interior wall of annulus fibrosus A. It is further envisioned that inflatable sealing member 34 is fabricated from a soft polymer with friction or sealing characteristics with the surrounding disc tissues.

In the inflated, sealing configuration, inflatable sealing member 34 pressurizes intervertebral disc I. Injectable polymer P is introduced into space S through needle 12 under pressure while inflatable sealing member 34 remains in its inflated sealing configuration, maintaining pressure of intervertebral disc I. As injectable polymer P is delivered to space S, the inflated, sealing configuration of inflatable sealing member 34 and corresponding pressurization of intervertebral disc I facilitates injection of polymer P deep into the tears and crevasses of damaged intervertebral disc I. This configuration provides improved disc filling and mechanical stabilization, as well as improved mechanical interlocking of polymer P with intervertebral disc I.

After a desired amount of injectable polymer P is delivered to space S, and optionally, the injectable polymer P sets or hardens to a desirable degree, the pressure to the inflatable sealing member 34 is discontinued and inflatable sealing member 34 returns to its uninflated, non-sealing configuration. Valve 24 is manipulated to release air from inflatable sealing member 34 via second lumen 36, such that inflatable sealing member 34 contracts and assumes its uninflated, non-sealing configuration. Inflatable sealing member 34 assumes its orientation in substantially flush alignment with exterior surface 32 of needle 12. Needle 12 is withdrawn from space S and intervertebral disc I. Post treatment procedures may be performed.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. An apparatus for introducing fluent therapeutic material through a needle and preventing or reducing leakage of the fluent therapeutic material back along the needle, the apparatus comprising:
a needle having a distal end for insertion through an entry site and a proximal end coupled to a source of pressurizable fluent therapeutic material,
the needle defining a first conduit through which the pressurized fluent therapeutic material is extruded; and
an expandable seal disposed about an exterior surface of the needle, the expandable seal having a non-expanded, non-sealing configuration to facilitate penetration of the needle and withdrawal of the needle, and an expanded, sealing configuration capable of being expanded during introduction of the fluent therapeutic material to prevent or minimize leakage of fluent therapeutic material through the entry site.

2. The apparatus of claim 1, wherein the expandable seal comprises an inflatable sealing member.

3. The apparatus of claim 2, wherein the needle defines a second conduit communicating with the inflatable sealing member and being configured to introduce a pressurized medium into the inflatable sealing member.

4. The apparatus of claim 3, wherein the second conduit is further configured to convey pressurized medium from the inflatable sealing member whereby the inflatable sealing member assumes a non-expanded, non-sealing configuration.

5. The apparatus of claim 1, wherein the expandable seal in its non-expanded, non-sealing configuration is disposed in a circumferential recess in an exterior surface of the needle.

6. The apparatus of claim 5, wherein an exposed surface of the expandable seal is substantially flush with the exterior surface of the needle.

7. The apparatus of claim 2, wherein the inflatable seal, in the expanded, sealing configuration has a substantially toroidal configuration.

8. The apparatus of claim 1, wherein the expandable seal, in its expanded, sealing configuration is dimensioned such that it occupies not more than about 20% of a volume contained by the annulus fibrosus.

9. The apparatus of claim 1, wherein the expandable seal, in its expanded, sealing configuration is dimensioned such that it does not occupy more than about 5% of a volume contained by the annulus fibrosus.

10. The apparatus of claim 1, wherein the needle further comprises at least one guide mark on its exterior surface to indicate a distance of insertion of the needle.

11. The apparatus of claim 1, wherein the needle further comprises a stop affixed to the exterior surface, the stop being configured to prevent over-insertion of the needle.

12. The apparatus of claim 2, wherein the inflatable sealing member is fabricated from at least one member selected from the group consisting of polyvinylchloride, polyethylene terephthalate (PET), nylon, polyurethane, silicone, thermoplastic rubber, nylon, and thermoplastic elastomer materials.

13. The apparatus of claim 1, wherein the fluent therapeutic material comprises at least one member selected from the group consisting of curable hydrogels, acrylic-based bone cements, pastes comprising bone particles, ceramic-based bone cements and natural polymers.

14. The apparatus of claim 3, wherein the pressurized medium is air.

15. A method for introducing fluent therapeutic material into the annulus fibrosus of an intervertebral disc, the method comprising the steps of:
a) inserting a needle through an entry site of the annulus fibrosus and into a nucleus pulposus of an intervertebral disc, the needle comprising:
(i) a distal end for insertion through the entry site and into the nucleus pulposus and a proximal end coupled to a source of fluent therapeutic material,
an inflatable seal disposed about an exterior surface of the needle, the inflatable seal having an uninflated, non-sealing configuration to facilitate penetration of the needle through the annulus fibrosus and withdrawal of the needle therefrom, and an inflated sealing configuration,
the needle defining a first conduit through which the fluent therapeutic material is introduced under pressure into the nucleus pulposus following insertion of the distal end through the annulus fibrosus; and
the needle defining a second conduit communicating with the inflatable seal, the second conduit being configured to introduce a pressurized inflating gas or fluid from a source of pressurized inflating gas or fluid, whereby the inflatable seal assumes its inflated sealing configuration, the second conduit also being configured to convey pressurized inflating gas or fluid from the inflated seal, whereby the seal reassumes its uninflated non-sealing configuration;
(ii) inflating the inflatable seal to a sealing configuration, preventing or minimizing leakage of the fluent therapeutic material through the entry site;
b) inflating the inflatable seal with the pressurized inflating gas or fluid, and expanding the inflatable seal to its inflated, sealing configuration such that the inflatable seal engages the annulus fibrosus to prevent or minimize flow of the fluent therapeutic material through the entry site during the introduction of the fluent therapeutic material into the space defined by displacement of the nucleus pulposus;
c) introducing the fluent therapeutic material under pressure into the space defined by displacement of the nucleus pulposus; and
deflating the inflatable seal and withdrawing the needle.

16. The apparatus of claim 3, wherein the needle defines a third conduit communicating with the inflatable sealing member and being configured to convey pressurized medium from the inflatable sealing member whereby the inflatable sealing member assumes a non-expanded, non-sealing configuration.

17. The method of claim 15, wherein the inflatable seal, in the expanded, sealing configuration has a substantially toroidal configuration.

18. The method of claim 17, wherein the inflatable seal, in its expanded, sealing configuration extends its circumference by not more than about 30% relative to the uninflated state.

19. The method of claim 15, wherein the inflatable seal, in its expanded, sealing configuration is dimensioned such that it does not occupy more than about 5% of a volume contained by the annulus fibrosus.

20. An apparatus for introducing fluent therapeutic material through an annulus fibrosus of an intervertebral disc, the apparatus comprising:
a needle having a distal end for insertion through an entry site of the annulus fibrosus and a proximal end coupled to a source of a pressurizable injectable polymer, the needle including a circumferential recess defined in an exterior surface thereof adjacent the distal end,
the needle defining a first lumen conduit through which the injectable polymer is introduced; and
an inflatable sealing member configured for disposal about the exterior surface of the needle, and in the circumferential recess the inflatable sealing member having a non-inflated, non-sealing configuration to facilitate penetration of the needle through the annulus fibrosus and withdrawal of the needle therefrom, an exposed surface of the inflatable sealing member being in a substantially flush alignment with the exterior surface of the needle in the non-inflated, non-sealing configuration, and
the inflatable sealing member further having an inflated, sealing configuration during introduction of injectable polymer, the inflatable sealing member having a substantially toroidal configuration and engaging the annulus fibrosus in its inflated, sealing configuration to prevent or minimize leakage of the injectable polymer through the entry site,
the needle further defining a second lumen communicating with the inflatable sealing member, the second lumen being configured to introduce a pressurized medium from a source of pressurizable medium into the inflatable sealing member and to convey the pressurized medium from the inflatable sealing member as the inflatable sealing member assumes its uninflated, non-sealing configuration,
whereby the inflatable sealing member assumes its inflated, sealing configuration during introduction of the injectable polymer under pressure into the annulus fibrosus and is dimensioned such that it does not occupy more than about 5% of a volume contained by the annulus fibrosus.
